(19) United States
(12) Patent Application Publication
(10) Pub. No.: US 2024/0175039 A1
(43) Pub. Date: May 30, 2024

(54) ENGINEERING PROBIOTIC FOR DEGRADING URIC ACID, AND CONSTRUCTION METHOD THEREFOR AND USE THEREOF

(71) Applicant: SHANDONG UNIVERSITY, Shandong (CN)

(72) Inventors: Yongzhen XIA, Shandong (CN); Luying XUN, Shandong (CN); Rui ZHAO, Shandong (CN)

(73) Assignee: SHANDONG UNIVERSITY, Shandong (CN)

(21) Appl. No.: 18/556,678

(22) PCT Filed: Mar. 7, 2023
(86) PCT No.: PCT/CN2023/074803

§ 371 Date: Oct. 23, 2023

(30) Foreign Application Priority Data
Feb. 14, 2022 (CN) 202210134048.6

(51) Int. Cl. C12N 15/72; A61K 35/741; A61P 19/06; C07K 14/47; C07K 14/805; C12N 9/06; C12N 9/08

(52) U.S. Cl. CPC C12N 15/72; A61K 35/741; A61P 19/06; C07K 14/47; C07K 14/805; C12N 9/0048; C12N 9/0065; C12N 2800/22; C12Y 111/01006

(57) ABSTRACT

Provided are an engineering probiotic for degrading uric acid, and a construction method therefor and the use thereof, which belongs to the field of biotechnology. The probiotic Escherichia coli Nissle 1917 is used as an original starting strain, and an exogenous gene is introduced into a genome thereof by means of genetic engineering technology for modification, such that uric acid can be efficiently and rapidly degraded; and it is proved by tests that the rapid degradation of uric acid can be achieved in the intestinal tract and blood of mice. Compared with current lactic acid bacteria for degrading uric acid in the market, the constructed engineering probiotic has a stronger degradation ability and a better treatment effect, and therefore has a good practical application value.

The present invention claims the priority of China patent application no. 202210134048.6, filed with the China Patent Office on Feb. 14, 2022, entitled “Engineering probiotic for degrading uric acid, and construction method therefor and use thereof”, the entire contents of which are incorporated herein by reference and form a part of the present invention.

### BACKGROUND OF THE INVENTION

### 1. Technical Field

The present invention belongs to the field of biotechnology and specifically relates to an engineering probiotic for degrading uric acid, and construction method therefor and use thereof.

### 2. Background Art

The disclosure of this background section is merely intended to increase the understanding of the overall background of the present invention and is not necessarily considered an admission or in any form to imply that the information constitutes prior art known to those of ordinary skill in the art.

Uric acid (UA) is a heterocyclic compound composed of an imidazole ring and a pyrimidine ring and is a class of intermediate products of purine metabolism. Humans and primates lack urate oxidase, an enzyme that metabolizes UA, so UA can only be excreted from the body as the final metabolic product of purine metabolism. However, if the metabolism of UA in the body is disturbed, it will cause gout. Gout is a relatively common joint inflammation. When the blood UA concentration exceeds the solubility saturation of urate, urate precipitates at the joints, which in turn causes inflammation. Clinical studies have found that kidney disease, cardiovascular disease, and diabetes are closely related to gout. With the continuous improvement of living standards, the incidence of gout has increased significantly compared to before, and the early symptom of gout—hyperuricemia—has affected 13.3% of the population. At present, there are mainly two types of treatments for gout and hyperuricemia: one is a low-purine diet, ensuring that the daily intake of purine does not exceed 400 mg; the other is to control the blood UA concentration by drugs, such as allopurinol, which can inhibit the activity of xanthine dehydrogenase and thus inhibit the production of UA. However, both methods have their drawbacks, and the treatment effect for gout is also relatively limited.

The excess UA in the human body is mainly excreted in two ways, about ⅔ (two-thirds) through the kidneys and ⅓ (one-third) through the intestines. At the same time, the blood UA concentration of mice with kidney removal did not increase significantly, and a class of transport proteins on the intestine: ABCG2, can transport UA to the intestine, and the activity of this protein has a very important impact on the blood UA concentration. It can be seen that intestinal UA metabolism is a very important pathway. The human intestine contains rich intestinal flora, and early studies also detected the presence of UA metabolites such as allantoin, allantoic acid, CO₂, etc. in the human body by isotope labeling methods, but the human body lacks urate oxidase, which cannot further metabolize UA, so it may be that the intestinal flora further metabolized UA. The human intestine is not a completely anaerobic environment as traditionally understood, but the oxygen concentration shows a gradient distribution. The closer to the small intestinal villi, the higher the oxygen concentration will be, dropping from about 40 mmHg to <1% mmHg.

At present, some people have detected a certain degree of reduction in blood UA concentration by oral administration of probiotics such as lactic acid bacteria and bifidobacteria, but the degree of reduction is limited. However, modifying the intestinal flora by synthetic biology methods has gradually become an important field of medical research. Escherichia coli Nissle 1917 is also a probiotic that has been widely used by humans to treat gastrointestinal problems such as gastroenteritis since 1917. Since Nissle 1917 does not disrupt the structure of the intestinal flora, and can also rapidly colonize in the intestine, it is currently used as an important strain for engineering therapy. Some people have constructed engineered bacteria through Escherichia coli Nissle 1917 to effectively metabolize phenylalanine and alleviate phenylketonuria by feeding mice and monkeys, among which an important enzyme LAAD activity requires oxygen, and also successfully expressed in the intestine. This indicates that the constructed engineered probiotics are hopeful for maintaining blood UA concentration, but the inventors found that recombinant probiotics for degrading UA are currently lacking in relevant applications.

### SUMMARY OF THE INVENTION

To overcome the above technical problems, the present invention first proposes constructing engineered probiotics to treat diseases related to elevated uric acid such as hyperuricemia and provides an engineered probiotic for degrading UA and its construction method and application. The present invention modifies the probiotic Escherichia coli Nissle 1917, as an original starting strain, by introducing exogenous genes into its genome using genetic engineering techniques, thereby enabling it to degrade UA efficiently and rapidly. Experimental results show that the engineered probiotic can also rapidly degrade UA in the intestine and blood of mice, and therefore have good practical application value.

To achieve the above technical objectives, the present invention adopts the following technical solutions:

In a first aspect, the present invention provides an engineered probiotic for degrading UA, wherein the probiotic is a derivative of Escherichia coli (E. coli), and wherein one or more exogenous genes selected from a urate oxidase gene, a urate transporter gene, a hemoglobin gene, and a catalase gene are integrated into its genome.

Wherein, the derivative of E. coli is specifically a derivative of E. coli Nissle 1917.

The urate oxidase gene is specifically PucL^{M} and PucM, whose nucleotide sequences are shown as SEQ ID NO: 1 and SEQ ID NO: 2, respectively; they act together, and in the present invention, they are named PucL^{M}M.

The urate transporter gene is specifically YgfU, whose nucleotide sequence is shown as SEQ ID NO: 3.

The hemoglobin gene is specifically Vhb, whose nucleotide sequence is shown as SEQ ID NO: 4.

The catalase gene is specifically KatG, whose nucleotide sequence is shown as SEQ ID NO: 5.

In a second aspect, the present invention provides a method for constructing the above-mentioned engineered probiotic for degrading UA, wherein the method comprises: introducing any one or more of the exogenous genes selected from urate oxidase gene, urate transporter gene, hemoglobin gene, and catalase gene into E. coli.

A third aspect of the present invention provides an application of the above-mentioned engineered probiotic in degrading UA.

It has been proved by experiments that the above-mentioned engineered probiotics of the present invention exhibit excellent performance in degrading UA in both oxygen-rich and hypoxic (such as oxygen-limited intestinal) environments, and therefore can be used as a therapeutic drug for diseases caused by high UA levels (such as gout, hyperuricemia, etc.).

Therefore, a fourth aspect of the present invention provides a pharmaceutical composition, wherein an active ingredient of the pharmaceutical composition comprises the above-mentioned engineered probiotic. The pharmaceutical composition can be used for treating related diseases caused by high UA levels (such as gout, hyperuricemia, etc.), and compared with the prior art, it has a stronger ability to degrade UA and better therapeutic effect.

Wherein, the pharmaceutical composition may further comprise one or more pharmaceutically acceptable carriers, excipients, and/or diluents.

A fifth aspect of the present invention provides a method for treating diseases related to elevated UA levels, wherein the method comprises administering to a subject a therapeutically effective dose of the above-mentioned engineered probiotic and/or pharmaceutical composition.

The beneficial technical effects of one or more of the above-mentioned technical solutions are as follows:

The present invention first proposes constructing engineered probiotics to treat diseases related to elevated uric acid levels such as hyperuricemia. The technical solution of the present invention is to use E. coli Nissle 1917 as an original starting strain and introduce exogenous genes including urate oxidase gene, urate transporter gene, hemoglobin gene, and catalase gene by genetic engineering technology, thereby constructing an engineered probiotic that can efficiently degrade UA. Compared with the lactic acid bacteria that degrade UA currently available on the market, the engineered probiotics constructed by the present invention have stronger degradation ability and better therapeutic effect and therefore have good practical application value.

### BRIEF DESCRIPTION OF THE DRAWINGS

The drawings accompanying the specification forming a part of the present invention serve to provide a further understanding of the present invention. The schematic embodiments of the present invention and their description are used to explain the present invention and do not constitute an improper limitation of the present invention.

FIG. 1: The schematic diagram of an engineered EcN strain for hyperuricemia therapy.

FIG. 2: The optimization of UA degradation by engineered EcN strain constructed by the present invention. (A-B) UA degradation by using crude enzymes (A) or whole cells (B) of engineered EcN expressing PucL^{T} in different plasmids under the control of different promoters. (C) UA degradation by EcN whole cells with PucL, PucL^{T}, and PucL^{M}. (D) UA degradation by EcN whole cells by co-expressing ygfU. The degradation curves were determined in HEPES buffer (pH=7.0) at OD₆₀₀=1.0 for whole cells or with proteins at 0.8 mg/mL for enzymatic assays. The UA degradation ability of these whole cells or crude enzymes was assayed at defined time intervals. Three parallel experiments were executed to obtain mean values and calculate STDEV. One-way analysis of variance (one-way ANOVA) method was used to calculate p values. Q values were calculated to obtain a false discovery rate (FDR). Q<0.05, marked with “*”; Q<0.01, marked with “**”; Q<0.001, marked with “***”. In the four graphs, only the Q values between the mean data of the two groups representing the fastest UA degradation rates were shown.

FIG. 3: Vhb and KatG in the engineered EcN strain constructed by the present invention facilitated the recombinant EcN strain for UA degradation under either normal oxygen or hypoxic conditions. (A) UA degradation by EcN strains under normal oxygen conditions. UA degradation under normal oxygen conditions was done in flasks with shaking. The ROS level (B) and DO level (C) were also detected. Three parallel experiments were executed to obtain mean values and STDEV. (D) UA degradation by EcN strains under hypoxic conditions, where the DO is 15% of the normal oxygen content in the medium. UA degradation under hypoxic conditions was done in a bioreactor with controlled DO. The strains were cultured, induced, and resuspended into HEPES buffer (50 mM, pH=7.0) at OD₆₀₀=1.0. UA degradation by whole cells was assayed. For the bioreactor experiment, error bars were calculated from the data obtained in three different batches. The student's t-test method was used to calculate the p-value for UA degradation curve. p<0.05, marked with “*”; p<0.01, marked with “**”, p<0.001, marked with “***”, p<0.0001, marked with “****”.

FIG. 4: The engineered recombinant EcN strain constructed by the present invention degraded UA in mice jejunum. In the test group, the optimized engineered EcN strain was orally administered to mice first (n=6). After 1 hour, the UA was orally administered to these mice. In the positive control group, only UA was orally administered. In the negative control group, neither UA nor EcN was administered. After another hour, the UA levels in the stomach (A), duodenum (B), and jejunum (C) were measured. Six parallel experiments were executed to obtain averages and calculate the STDEV. The one-way ANOVA method was used to calculate the p-value. The Q values were calculated to obtain FDR. Q>0.05, marked with “NS”; Q<0.0001, marked with “****”.

FIG. 5: Therapeutic effect of using the engineered EcN strains via oral administration in the UA-injection hyperuricemia mice in the present invention. (A) The serum UA concentrations in mice after the intravenous injection of UA. It is named the UA-injection group. (B-D) 2×10¹⁰ CFU of the indicated engineered EcN strains containing the UA degradation genes with or without vhb and katG were orally administered once a day for 5 days in mice. Then UA was intravenously injected 1 hour after the last time of intragastric administration of the EcN strains. The serum UA concentrations were determined (B-C). The serum concentrations of H₂O₂ were determined (D). Six parallel experiments were executed to obtain averages and calculate STDEV. In graph B, the one-way ANOVA method was used to calculate the p-value. The Q values were calculated to get the FDR. Q>0.05, marked with “ns”; Q<0.0001, marked with “****”. Only the Q values between the mean data of the two groups representing the fastest UA degradation rates were shown. In graphs, C&D, the student's t-test method was used to calculate p values. p>0.05, marked with “ns”; p<0.05, marked with “*”; p<0.01, marked with “**”; p<0.001, marked with “***”.

FIG. 6: UA degradation by the engineered EcN strain in buffer, serum, and mice blood samples in the present invention. (A) UA degradation by the engineered EcN strains in HEPES buffer (50 mM, pH=7.0). (B) UA degradation by the engineered EcN strains in mice serum. (C) The degradation ability of engineered EcN strain in the mixed blood of young mice (ages at 6 weeks old, mixed blood samples=6). (D) The degradation ability of engineered EcN strain in the mixed blood of old mice (ages at 12 weeks old, mixed blood samples=6). The indicated engineered EcN strain was added to degrade UA in the sample, and then UA was added in several rounds. Three parallel experiments were executed to obtain mean values and to calculate STDEV.

FIG. 7: Therapeutic effect of using the engineered EcN strains via intravenous administration in UA-injection hyperuricemia mice in the present invention. The indicated amount of engineered EcN strains were injected. Then, the UA injection method was used to induce hyperuricemia in mice. The serum UA levels in different groups were detected at defined time intervals (A, B&E). The serum concentrations of H₂O₂ were also determined (F). The time intervals between engineered strain injection and UA injection were either 0 hours (A) or 10 hours (B, E&F). (C) The survival curves of mice in the two groups that were injected with two different amounts of engineered EcN strains were given. (D) Body weight of the mice in the two groups that were either injected with 5×10⁸ CFU engineered EcN strain or the same volume of saline (control). Six parallel experiments were executed to obtain averages and calculate the STDEV. For data in graphs A&B, p values were calculated using one-way ANOVA method. Q values were calculated to obtain FDR. Q<0.05, marked with “*”; Q<0.01, marked with “**”; Q<0.001, marked with “****”; Q<0.0001, marked with “****”. For the data in graphs E&F, p values were calculated using student's t-test method. p>0.05, marked with “ns”; p<0.05, marked with “*”; p<0.01, marked with “**”; p<0.001, marked with “***”.

### DETAILED DESCRIPTION OF THE EMBODIMENTS

It should be noted that the following detailed descriptions are exemplary and intended to provide further explanation of the present invention. Unless otherwise indicated, all techniques and scientific terminology used in this document have the same meaning as understood by those skilled in the art to which the present invention belongs.

It should be noted that the terminology used here is only to describe specific embodiments, and not intended to limit the exemplary embodiments of the present invention. As used herein, unless otherwise specified in context, the singular form also includes the plural form. Furthermore, it should be understood that when the terms “including” and/or “comprising” are used in this specification, they indicate the existence of features, steps, operations, devices, components, and/or combinations thereof. It should be understood that the scope of protection of the present invention is not limited to the specific embodiments described herein; it should also be understood that the terms used in the embodiments of the present invention are to describe specific embodiments, and not intended to limit the scope of protection of the present invention.

In a typical specific embodiment of the present invention, an engineered probiotic that degrades UA is provided. The probiotic is derived from E. coli, and one or more exogenous genes selected from a urate oxidase gene, a urate transporter gene, a hemoglobin gene, and a catalase gene are integrated into its genome.

Wherein, the derivative of E. coli is specifically a derivative of E. coli Nissle 1917.

The urate oxidase gene is specifically PucL^{M} and PucM, whose nucleotide sequences are shown as SEQ ID NO: 1 and SEQ ID NO: 2, respectively; they act together, and in the present invention, they are named PucL^{M}M.

The urate transporter gene is specifically YgfU, whose nucleotide sequence is shown as SEQ ID NO: 3.

The hemoglobin gene is specifically Vhb, whose nucleotide sequence is shown as SEQ ID NO: 4.

The catalase gene is specifically KatG, whose nucleotide sequence is shown as SEQ ID NO: 5.

In a specific embodiment of the present invention, the genotype of the engineered probiotic is EcN::pMCS2-Ptrc-pucL^{M}M-vhb-ygfU-katG.

In another specific embodiment of the present invention, a method for constructing engineered probiotics for degrading UA is provided. The method comprises: introducing any one or more of the above-mentioned exogenous genes selected from a urate oxidase gene, a urate transporter gene, a hemoglobin gene, and a catalase gene into Escherichia coli.

Specifically, the method comprises: constructing a recombinant expression vector and transferring the recombinant expression vector into E. coli Nissle 1917 for expression.

The recombinant expression vector is obtained by effectively linking any one or more of the genes in the above-mentioned genes to an expression vector, wherein the expression vector is any one or more of a viral vector, plasmid, phage, phagemid, cosmid, fosmid, phage or artificial chromosome; the virus vector may include an adenovirus vector, retrovirus vector or adeno-associated virus vector, and the artificial chromosome includes a bacterial artificial chromosome (BAC), a vector derived from phage P1 (PAC), a yeast artificial chromosome (YAC) or a mammalian artificial chromosome (MAC); the preferred vector is a plasmid of bacteria, and in a specific embodiment of the present invention, the bacterial plasmid is pBBR1MCS-2.

The urate oxidase gene is specifically identified as PucL^{M} and PucM, whose nucleotide sequences are as shown in SEQ ID NO: 1 and SEQ ID NO: 2, respectively; they act together, and in the present invention, they are named PucL^{M}M.

The urate transporter gene is specifically YgfU, whose nucleotide sequence is shown as SEQ ID NO:3.

The hemoglobin gene is specifically Vhb, whose nucleotide sequence is shown as SEQ ID NO:4.

The catalase gene is specifically KatG, whose nucleotide sequence is shown as SEQ ID NO:5.

Furthermore, the above-mentioned gene is expressed using a Ptrc promoter. In a specific embodiment of the present invention, the recombinant expression vector may be pBBR1MCS-2-Ptrc-pucL^{M}-pucM-ygfU-vhb-katG (whose sequence is shown as SEQ ID NO: 6).

The transformation method includes biologically acceptable direct transformation methods (including gene gun method, electroporation method, ultrasound method, microinjection method, and PEG method) and indirect transformation methods (including DNA viral vector-mediated method and agrobacterium-mediated method). Preferably, the electroporation method is used. In a specific embodiment of the present invention, the recombinant expression vector is electroporated into host bacteria by using a 2.5 kV pulse.

In another specific embodiment of the present invention, the application of the above-mentioned engineered probiotics in the degradation of UA is provided.

It has been proved by experiments that the above-mentioned engineered probiotics of the present invention exhibit excellent performance in degrading UA in both oxygen-rich and hypoxic (such as oxygen-limited intestinal) environments, and therefore can be used as a therapeutic drug for diseases caused by high UA levels (such as gout, hyperuricemia, etc.).

Therefore, in another specific embodiment of the present invention, a pharmaceutical composition is provided, wherein an active ingredient of the pharmaceutical composition comprises the above-mentioned engineered probiotic. The pharmaceutical composition can be used for treating diseases caused by high UA levels (such as gout, hyperuricemia, etc.), and compared with the prior art, it has a stronger ability to degrade UA and a better therapeutic effect.

Wherein, the pharmaceutical composition may further comprise one or more pharmaceutically acceptable carriers, excipients, and/or diluents.

In another specific embodiment of the present invention, the pharmaceutical composition comprises one or more pharmacy or food-acceptable excipients. The excipients used may be solid or liquid. Solid forms of the formulation include powders, tablets, dispersible granules, capsules, pills, and suppositories. Powders and tablets may contain about 0.1% to 99.9% of the active ingredient. Suitable solid excipients may be magnesium carbonate, magnesium stearate, talc, sugar, or lactose. Tablets, powders, pills, and capsules are solid dosage forms suitable for oral administration. Liquid forms of the formulation include solutions, suspensions, and emulsions, exemplary of which are non-enteral injectable solutions, water-propylene glycol solutions, or oral solutions containing sweeteners and contrast agents. In addition, it can also be made into injectable ampoules, injectable lyophilized powders, large-volume infusions, or small-volume infusions.

In another specific embodiment of the present invention, the pharmaceutical composition is a solid oral formulation, liquid oral formulation, or injectable formulation. Additionally, pharmaceutical administration can be performed on both humans and non-human mammals, such as mice, rats, guinea pigs, rabbits, dogs, monkeys, chimpanzees, and the like. Experimental results using mice following the present invention have demonstrated that UA can be rapidly and effectively reduced regardless of whether it is administered through intravenous injection or intragastric administration.

In another specific embodiment of the present invention, the pharmaceutical composition is in the form of tablets, dispersible tablets, enteric-coated tablets, chewable tablets, orally disintegrating tablets, capsules, sugar-coated tablets, granules, dry powder formulations, oral liquid formulations, injectable ampoules, injectable lyophilized powders, large-volume infusions, or small-volume infusions.

In another specific embodiment of the present invention, a method for treating diseases related to elevated UA levels is provided, wherein the method comprises administering to a subject a therapeutically effective dose of the above-mentioned engineered probiotic and/or pharmaceutical composition.

The term “subject” refers to an animal that is already a subject of treatment, observation, or experimentation, preferably a mammal, and most preferably a human. The term “therapeutically effective dose” refers to an amount of the engineered probiotic or pharmaceutical composition of the present invention that can elicit a biological or medical response in a tissue system, animal, or human that is sought by a researcher, veterinarian, physician, or other clinician, which includes alleviating or partially alleviating the symptoms of the disease, syndrome, condition, or disorder being treated.

It must be recognized that the optimal dosage and interval of administration of the active ingredient of the present invention are determined by its properties and external conditions such as the form, route, and site of administration and the specific mammal being treated, and this optimal dosage can be determined by conventional techniques. It must also be recognized that the optimal course of treatment, i.e., the daily dose of the compound for a given period, can be determined by methods well-known in the art.

To better illustrate the purpose, technical solution, and advantages of the present invention, the following will further describe the present invention concerning specific embodiments.

### EXAMPLE

### Methods

### I. Acquisition of Genes

The pucL (Gene ID: 936669) and pucM (Gene ID: 937977) genes from Bacillus subtilis were codon-optimized and synthesized by Beijing Genomics Institute (BGI, Beijing). Asp-214 and Gln-438 of PucL were mutated to Val and Arg, respectively. The mutated pucL was named as pucL^{M}, and pucL^{M} was codon-optimized and synthesized by Beijing Genomics Institute (BGI, Beijing). The vhb gene (Protein ID: WP_019959060.1) from Vitreoscilla sp. C1 was codon-optimized and synthesized by Beijing Genomics Institute (BGI, Beijing). The ygfU (Gene ID: 949017) and the katG (Gene ID: 948431) genes were PCR amplified from E. coli MG1655 genomic DNA.

### II. Strains and Vectors

The construction of the plasmid was performed using Escherichia coli XL1-Blue MRF′ as a host strain and the pBBR1MCS-2 plasmid as a vector. The pucL^{M} pucM ygfU, vhb, and katG genes were expressed under the control of the Ptrc promoter. Subsequently, the plasmid pBBR1MCS-2-Ptrc-pucL^{M}-pucM-ygfU-vhb-katG was electroporated into E. coli Nissle 1917 using Eppendorf Eporator™ (BIO-RAD, Irvine, USA) with 2.5-kV pulses.

The complete plasmid sequence is shown as SEQ ID NO: 6.

### III. Reagents, Enzymes, and Related Kits

Antibiotic (kanamycin sulfate) purchased from Sangon Biotech (Shanghai) Co., Ltd.

Various chemical reagents were all analytical grade and purchased from Sigma (Shanghai) Company.

Various restriction endonucleases and DNA ligases were purchased from Thermo Company.

DNA marker purchased from Beijing TransGen Company. The plasmid extraction kit and gel recovery kit were purchased from Omega Company.

### IV. Detection of Bacterial Growth

Bacterial growth was detected using a UV-visible spectrophotometer (UV-1800, Shimadzu, Japan) to directly measure the absorbance at 600 nm.

### V. Cultivation, Induction, Collection, and Preservation of Engineered Bacteria

The recombinant strains were inoculated into 300 mL triangular flasks, with the addition of 50 mL fresh LB medium containing 50 μg/mL kanamycin sulfate and shaken overnight at 37° C. (200 rpm). The following day, the well-grown engineered bacteria were inoculated into 2 L triangular flasks. 500 mL fresh LB medium was added, and the initial inoculation OD₆₀₀ was 0.05. Shaking was continued at 37° C. (200 rpm). Until the OD₆₀₀ of the bacterial cultures reached ˜0.6, 2 mM isopropyl-β-D-thiogalactopyranoside (IPTG) was added, and the engineered strains were shaken at 30° C. (140 rpm) and further cultivated for 24 hours. After induction, the bacterial strains were collected by centrifugation at 6000 rpm for 10 minutes at 4° C., washed once with prepared phosphate buffer (2.28 g/L KH₂PO₄, 14.5 g/L K₂HPO₄, 15% glycerol, pH 7.5), and then concentrated in the buffer to a concentration of 5×10¹⁰ CFU/mL. The strains were stored at −80° C. until the day of analysis.

### VI. Method for UA Determination

Four methods were used to determine the concentration of UA in different cases.

(1) UA was measured in a buffer solution using a spectrophotometer by directly measuring the absorbance at 293 nm. Firstly, UA standard solutions of 0, 10 μM, 15 μM, 20 μM, 30 μM, 40 μM, 50 μM, 60 μM, and 70 μM were prepared to plot an UA concentration standard curve, obtaining a curve R2 of 0.9997. Subsequently, appropriate dilutions were performed for measurement, and UA concentration was determined.

(2) When UA and its degradation product (S)-allantoin were determined in complex media, an HPLC method was adopted. Briefly, a C18 reverse phase HPLC column (ODS-A, 250×4.6×4.6 mm, YMC) was used. The mobile phase was selected with Solvent A (2.5 mM NH₄H₂PO₄ buffered to pH of 3.5 with phosphoric acid) and Solvent B (5% solvent A and 95% methanol). The C18 reverse phase HPLC column was first pre-equilibrated with 100% Solvent A and 0% Solvent B. The column was eluted with the following gradients of Solvent B: 0% from 0 to 3.5 min; 40%-80% from 3.5 to 11.5 min; 80%-100% from 11.5 to 15.3 min; 100%-0% from 15.3 to 18 min; 0% from 18 to 22 min. The flow rate was 1.0 mL/min. UA and allantoin were detected by using an HPLC device (LC-20AT, Shimadzu, Japan) with a diode array detector (SPD-20A, Shimadzu, Japan) at an absorbance of 205 nm. Normally, the allantoin absorption peak appears around 3.2 minutes, and the UA absorption peak appears around 9.8 minutes. UA standard solutions of 100 μM, 200 μM, 500 μM, 750 μM, 1000 μM, 1500 μM were prepared, and UA standard curves were plotted with HPLC peak areas, with a standard curve R2 of 0.9973; allantoin standard solutions of 10 μM, 20 μM, 50 μM, 100 μM, 200 μM, 500 μM, 750 μM, 1000 μM were prepared, and allantoin standard curves were plotted with HPLC peak areas, with a standard curve R2 of 0.9999.

(3) When low concentrations of UA (<180 μM) were determined in blood samples, a UA assay kit (Solarbio, Beijing) was used according to the manual instruction

(4) When UA concentration in the blood sample was higher than 180 μM, a UA meter PD-G001-3-P (BeneCheck, Beijing, China) was used, which could be operated with simple procedures and low sample volume (<5 μL), following the manual instructions.

### VII. Detection of UA Under Aerobic Conditions

The recombinant strain was inoculated in a 300 mL triangular flask containing 50 mL fresh LB medium supplemented with 50 μg/ml kanamycin sulfate. The initial inoculation OD₆₀₀ was 0.05, and the culture was shaken at 37° C. (200 rpm). When the OD₆₀₀ of the culture reached ˜0.6, 2 mM isopropyl-β-D-thiogalactoside (IPTG) was added, and the culture was shaken at 30° C. (200 rpm) and further incubated for 24 hours. The cells were collected by centrifugation at 4° C., 6000 rpm for 10 minutes, and washed once with HEPES buffer (pH 7.0, 50 mM). The OD₆₀₀ of the induced engineered cells was adjusted to 1.0. The 20 mL resuspended induced engineered cells were transferred directly to a 50 mL centrifuge tube. 1 mM uric acid was added to initiate the reaction, and the tubes were incubated at 37° C. with shaking (200 rpm) for 60 minutes. Samples were taken every 15 minutes. After centrifugation at 13000 rpm for 3 minutes, the concentration of UA in the supernatant was determined using spectrophotometry.

### VIII. Detection of UA Under Limited Oxygen Conditions

Recombinant cells were cultured and harvested in the same manner as preparing whole cells under aerobic conditions. The whole cells were suspended in 20 mL of Brain Heart Infusion Broth (HB8297-1, Hopebio). 700 mL of Brain Heart Infusion Broth was added to a 1.4-L Multifors parallel bioreactor (Infors HT, US) and autoclaved together with the fermenter. 10 mL of Fetal Bovine Serum (04-121-1A, Biological Industries) was added to the bioreactor as a supplemental nutrient before adding the engineered cell. Following the addition of 2.0 mM IPTG and 50 μg/mL kanamycin, finally, a defined volume of EcN-engineered bacteria was inoculated to control the initial OD₆₀₀=1.0. The cells were grown for an additional hour with a dissolved oxygen (DO) level equal to 15% of the normal condition. Both eutrophic Brain Heart Infusion Broth and low oxygen were used to simulate the intestinal environment. Subsequently, 1 mM UA was added to the bioreactor to initiate UA metabolism under these oxygen-limiting conditions. Samples were collected at specified time intervals. After centrifugation, 10 μL of the supernatant was used for UA and allantoin measurement using the HPLC method.

### IX. Determination of UA Degradation Ability of Engineered EcN Strain in Buffer Solution and Mouse Serum

In buffer solution: Engineered EcN strains were added to HEPES buffer (50 mM, pH 7.0) to adjust the number of whole cells to 1×10⁹ CFU/mL, 1×10⁸ CFU/mL and 1×10⁷ CFU/mL. A certain amount of UA solution was added until the final concentration of UA in the system reached 500 μM. After reacting for some time under 37° C., samples were taken at regular intervals to determine the change of UA within 2 hours.

In mouse serum: (1) serum from mice of 4 to 6 weeks old was purchased from YZYBIO (Henan, China). Engineered EcN strains were added to mouse serum to adjust the number of whole cells to 1×10⁹ CFU/mL, 1×10⁸ CFU/mL, and 1×10⁷ CFU/mL. A certain amount of UA solution was added until the final concentration of UA in the system reached 650 μM. After reacting for some time under 37° C., samples were taken at regular intervals to determine the change of UA within 2 hours.

(2) Serum from 6-week-old and 12-week-old mice was taken to simulate human UA concentration in vivo with adding UA, 1×10⁸ CFU/mL engineered EcN strains were added, reacted for a while under 37° C., samples were taken at regular intervals, and the change of UA was determined within a period. After UA was completely degraded, a certain amount of UA was added again for measurement, and this was repeated twice.

### X. Mouse Feeding

Five-week-old (20±2 g) Kunming mice were purchased from Henan Skbex Biotechnology Company. Mice were housed in a room (22±2° C.) with 65% humidity and a 24-hour light-dark cycle. Mice were fed a standard diet. Before the experiments were executed, mice were habituated for a week. Mice were in good health condition at the beginning of the experiments. Mice were randomly divided into groups (n=6) for subsequent experiments. All animal experiments followed the National Institutes of Health Guide for the Care and Use of Laboratory Animals (NIH Publications No. 8023, revised 1978.) and were approved by the Animal Ethics Committee of Shandong University.

### XI. Detection of UA Degradation Ability of Engineered Bacteria in Mouse Intestine

Eighteen 6-week-old male Kunming mice were divided into three groups (n=6). The test group was administered with 200 μL engineered EcN strain by intragastric administration (5×10¹⁰ CFU/mL). Both the positive control group and negative control group were intragastrically administered with the same volume of phosphate buffer (2.28 g/L KH₂PO₄, 14.5 g/L K₂HPO₄, 15% glycerol, pH 7.5). One hour later, mice in the test group and the positive control group were intragastrically administered with 1 mL of 20 mM UA. The mice in the negative control group were administered with the same volume of saline. After 30 min, mice were anesthetized by ethyl ether and sacrificed. The stomach and small intestine segments such as duodenum, jejunum, etc. were collected, and different organs were frozen in liquid nitrogen and stored at −80° C. for further analysis.

The stomach, duodenum, jejunum, and ileum tissues from the three groups were cut, weighed (controlled at around 0.1 g), and homogenized in 1 mL PBS (Phosphate-buffered saline) (pH=7.4) by using a homogenizer (Tissueprep TP-24, Gering, Beijing, China) at a speed of 4 for 5 minutes. After homogenization, the lysis mixtures were centrifuged (10 min, 10000 rpm at 4° C.) and kept at 4° C. for measurement. The supernatants were utilized to analyze the UA and protein concentrations. The UA concentration was determined by using the UA assay kit. The protein concentration was determined by using OD₂₈₀, which was detected by a microvolume spectrophotometer (Kaiao Tech, Beijing, China). After measurement, the UA level in the intestine was calculated based on the UA/protein concentration.

### XII. New Method for Constructing Mouse Hyperuricemia

Twelve 6-week-old male Kunming mice were divided into three groups (n=6). The test group was injected with 70 mg/kg of UA solution via the tail vein. UA injections were used to induce physiologically relevant serum uric acid (SUA) levels in humans. The control group was only injected with saline via the vein. UA concentrations were measured using a UA meter. Blood samples collected from the mouse tail at specified time intervals were measured.

### XIII. Measurement of UA Degradation Ability of Engineered Bacteria Under the New Mouse Hyperuricemia Method

The Engineered EcN strain was used to treat the hyperuricemia mice by either intragastric or intravenous administration.

(1) For intragastric treatment, eighteen 6-week-old male Kunming mice were divided into three groups: two test groups and a control group (UA-injection group). Defined amounts of the engineered EcN strains and EcN strains with the empty vector were intragastrically administered into mice in the two test groups. The control group mice were intragastrically administered with an equivalent amount of glycerol-phosphate buffer. All mice in the three groups were intravenously injected with the UA solution at 70 mg/kg. Mice in the intragastric administration group were orally administrated with 2×10¹⁰ CFU of the EcN cells of two strains for 5 consecutive days, UA solution was intravenously injected 1 hour after the last bacterial administration at day 5.

(2) For intravenous treatment, eighteen 6-week-old male Kunming mice were divided into three groups: two test groups and a control group (UA-injection group). Defined amounts of engineered EcN strains and EcN strains with the empty vector were injected into the mice in the two test groups. The control group mice were injected with an equivalent amount of glycerol-phosphate buffer. The mice receiving intravenous injection of EcN strain were first injected with bacteria, and then the UA solution was injected into the mice 10 hours after the bacterial injection.

For both treatments, the UA concentration was determined with a UA meter from blood samples taken from mouse tails at defined time intervals.

### Results

### I. UA Degradation Pathways Engineered in EcN

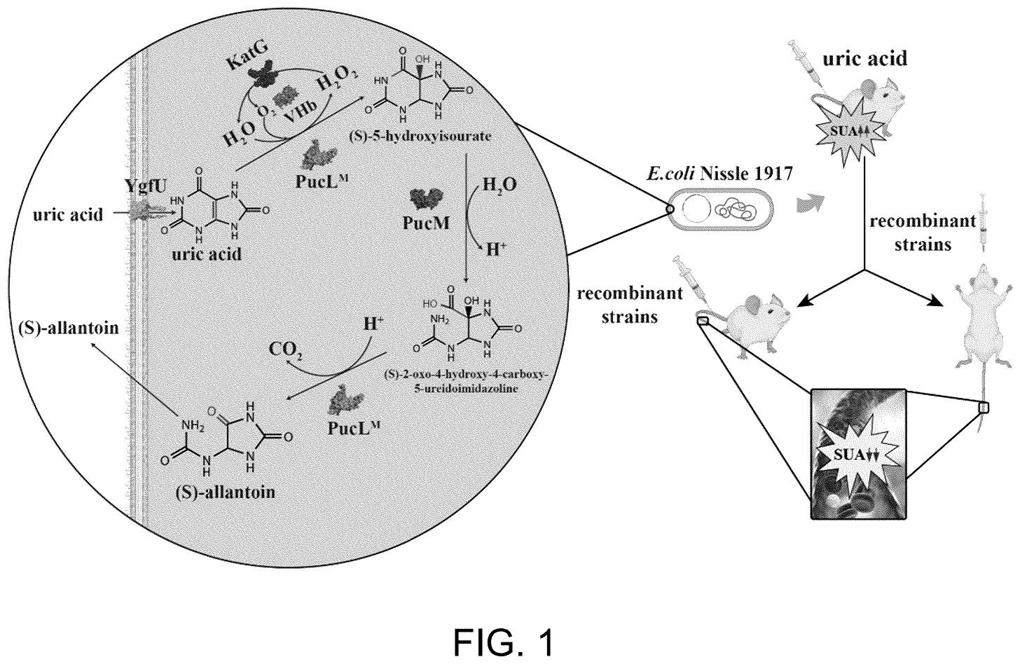

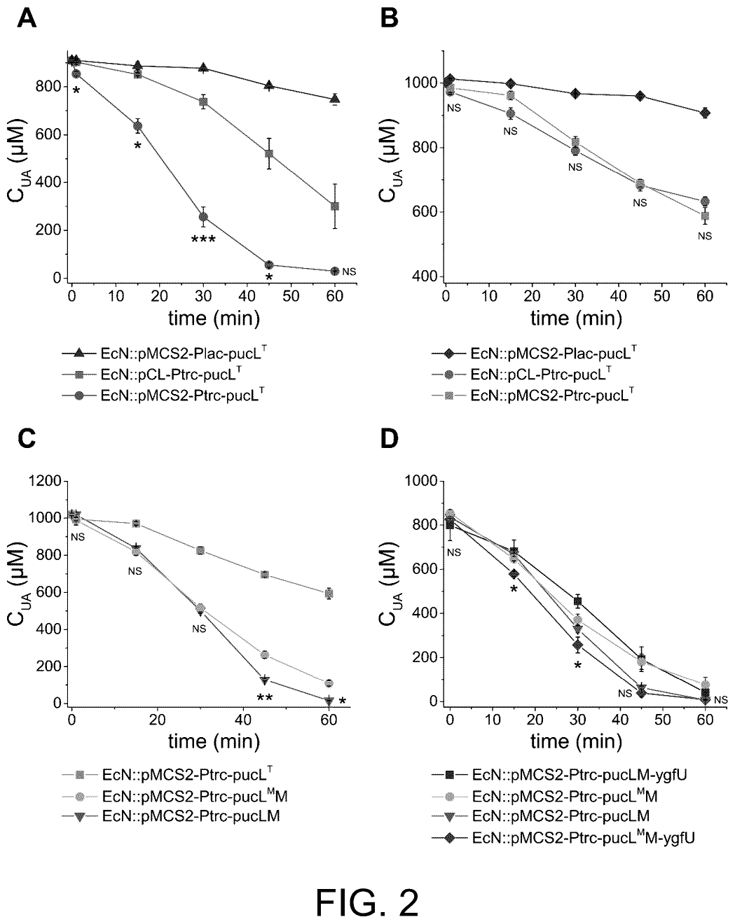

The UriC domain of PucL (PucL^{T}) catalyzes step 1 in the UA degradation (FIG. 1), and steps 2 and 3 can occur spontaneously. After codon optimization of PucL^{T}, two plasmids (pBBR1MCS-2 and pCL1920) and two promoters (Ptrc and Plac) were tested for the ability to express PucL^{T}. EcN::pMCS2-Ptrc-pucL^{T} with the gene encoding pucL^{T} under the control of the Ptrc promoter in pBBR1MCS-2 offered the highest UA degradation ability in the cell extract (A of FIG. 2); however, the same result was not reflected with whole-cell assays (B of FIG. 2).

Then, we performed codon optimization on the complete PucL and PucM, resulting in the strain EcN::pMCS2-Ptrc-pucL^{M}. We further compared the UA degradation ability of EcN::pMCS2-Ptrc-pucL^{M} and EcN::pMCS2-Ptrc-pucL^{T}. For both strains' cell extracts, the Vₘₐₓ of the two systems was similar, but the Kₘ of PucLM was significantly reduced (Table 1), indicating that cells with the complete pathway could effectively utilize UA at low concentrations. The UA degradation ability of the whole cell was greatly increased (FIG. 2C). The activity of PucL^{T} can be enhanced by the D44V and Q268R mutations. We directly mutated two sites in the complete PucL (PucLM) and co-expressed them with PucM in EcN (EcN::pMCS2-Ptrc-pucL^{M}M). Determined by using cell extracts, the Vₘₐₓ of PucL^{M}M increased by approximately 1.8 times compared to PucLM. However, the Kₘ of PucL^{M}M also increased by approximately 2.0 times (Table 1). Due to the increase in Kₘ, the UA degradation ability of the whole cell of EcN::pMCS2-Ptrc-pucL^{M}M was weakened when the concentration of UA was low (FIG. 2C).

TABLE 1

| | | Vₘₐₓ |
|---|---|---|
| Recombinant EcN | Kₘ (μM) | (μM/mg/min) |
| EcN::pMCS2-Ptrc-pucL^{T} | 61.03 | 12.35 |
| EcN::pMCS2-Ptrc-pucLM | 17.18 | 13.88 |
| EcN::pMCS2-Ptrc-pucL^{M}M | 52.54 | 38.63 |

When the UA transporter protein YgfU of E. coli was co-expressed with PucL^{M}M, the UA degradation ability of the whole cell of EcN::pMCS2-Ptrc-pucL^{M}M-ygfU was enhanced (FIG. 2D). Surprisingly, overexpression of YgfU significantly weakens the degradation ability of EcN::pMCS2-Ptrc-pucL^{M}-ygfU (FIG. 2D). Therefore, the fastest UA degradation strain EcN::pMCS2-Ptrc-pucL^{M}M-ygfU was used for next step optimization.

### II. UA Degradation Ability and Reduced Oxidative Stress Level of Engineered Strains Under Hypoxia or Anoxia Conditions

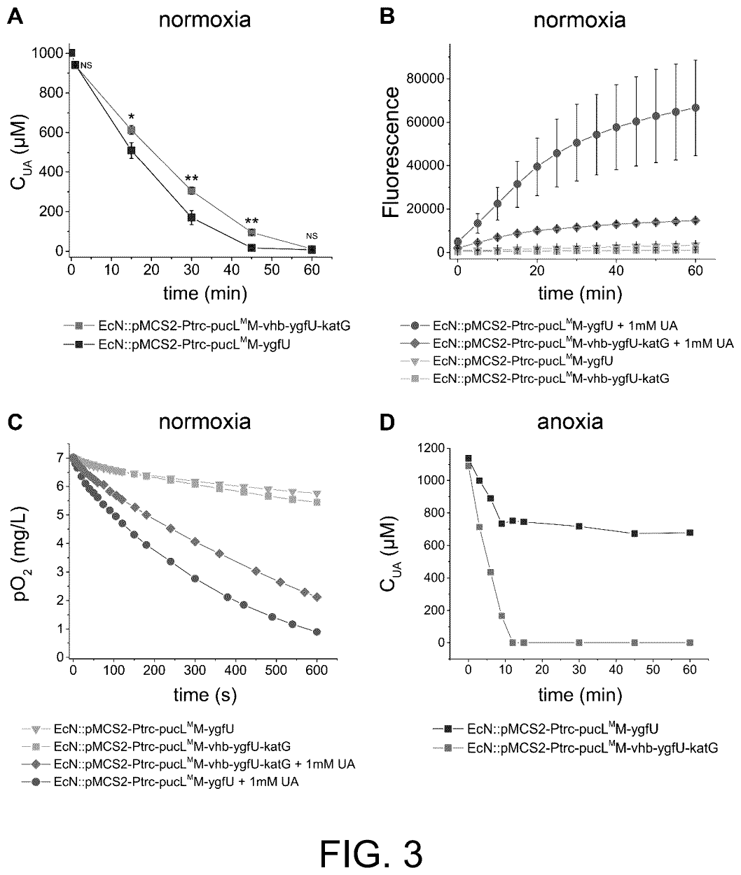

The katG gene encoding E. coli catalase was used to convert H₂O₂ produced by PucL into O₂, and Vhb encoding a bacterial hemoglobin protein from Vitreoscilla sp. C1 was applied to allow the engineered strain to degrade UA at low 02 levels. Therefore, the constructed EcN::pMCS2-Ptrc-pucL^{M}M-vhb-ygfU-katG produces PucL^{M}M, Vhb, YgfU, and KatG in the same bacterium. The rate of UA degradation by EcN::pMCS2-Ptrc-pucL^{M}M-vhb-ygfU-katG is slightly slower than that of EcN::pMCS2-Ptrc-pucL^{M}M-ygfU under normal oxygen conditions (A of FIG. 3). However, fewer reactive oxygen species (ROS) are generated (B of FIG. 3). As expected, the presence of KatG and Vhb also restores oxygen in the system (C of FIG. 3). When the dissolved oxygen in the culture medium is limited to around 15% of normal dissolved oxygen conditions, the strain EcN::pMCS2-Ptrc-pucL^{M}M-vhb-ygfU-katG can degrade more UA in a short period (D of FIG. 3). This modification step facilitated the utilization of oxygen and relieved possible damage caused by by-products in the UA degradation process.

### III. EcN::pMCS2-Ptrc-pucL^{M}M-Vhb-ygfU-katG Reduced Serum UA Level in Hyperuricemia Mice by Transplanting in the Gut

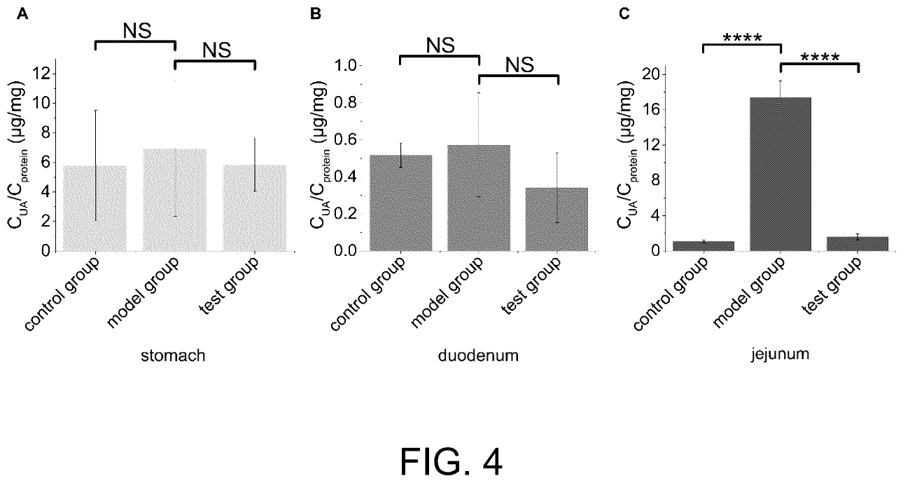

To check if our engineered strain could degrade UA efficiently in the gut, the test group was intragastrically administered with both UA and the induced engineered EcN strains, the model group was intragastrically administered with only UA, and the control group was intragastrically administered with saline. After 30 min, UA concentrations in the stomach and duodenum of the test group were not significantly different among the three groups (A&B of FIG. 4), but the UA level in the jejunum of the model group was much higher than those of the test groups and control groups (C of FIG. 4).

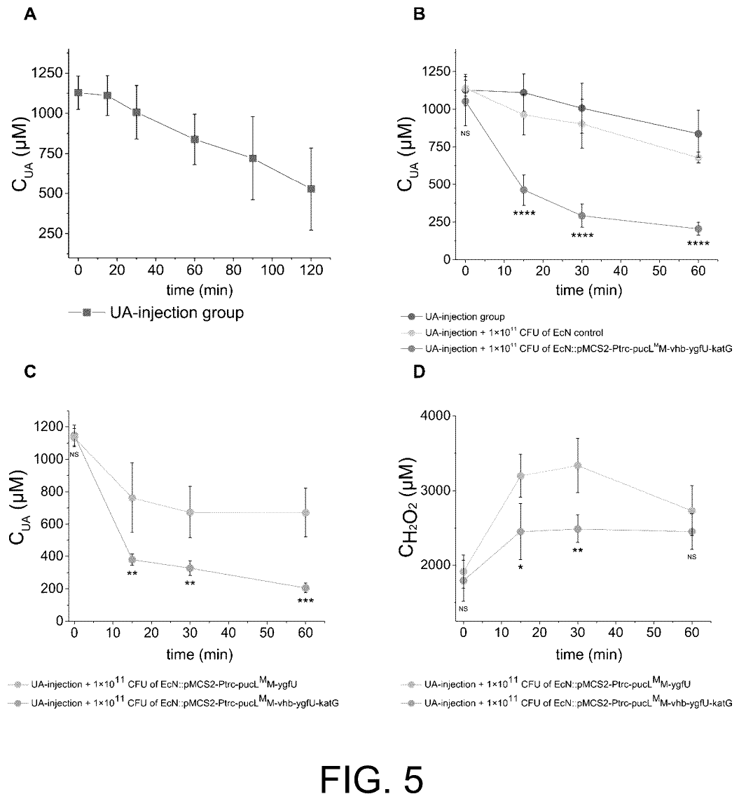

To increase serum UA in mice to levels comparable to that in humans, a UA solution was injected into the blood vessel at a dose of 70 mg/kg. Serum UA was promoted up to ˜1 mM, and the concentration gradually decreased over time (A of FIG. 5). No mouse was dead due to this treatment. This method was referred to as the UA injection method.

EcN::pMCS2-Ptrc-pucL^{M}M-vhb-ygfU-katG was used to treat UA-injection hyperuricemia mice. Engineered EcN strains were intragastrically administered to mice. Compared to the group administered intragastrically with EcN strain containing empty plasmid and the UA-UA-injection control group, the group of mice orally administered with 1×10¹¹ CFU of EcN strain containing uric acid degradation genes showed a significant decrease in blood UA levels (B of FIG. 5). We also tested the effect of VHb and KatG given in the test mice with EcN::pucL^{M}M-vhb-ygfU-katG and EcN::pucL^{M}M-ygfU. The overexpression of vhb and katG in EcN was confirmed to accelerate the rates of UA degradation and H₂O₂ removal in the UA-injection hyperuricemia mice (C&D of FIG. 5).

### IV. Intravenous Administration of EcN::pMCS2-Ptrc-pucL^{M}M-Vhb-ygfU-katG Relieved Hyperuricemia in the Test Mice

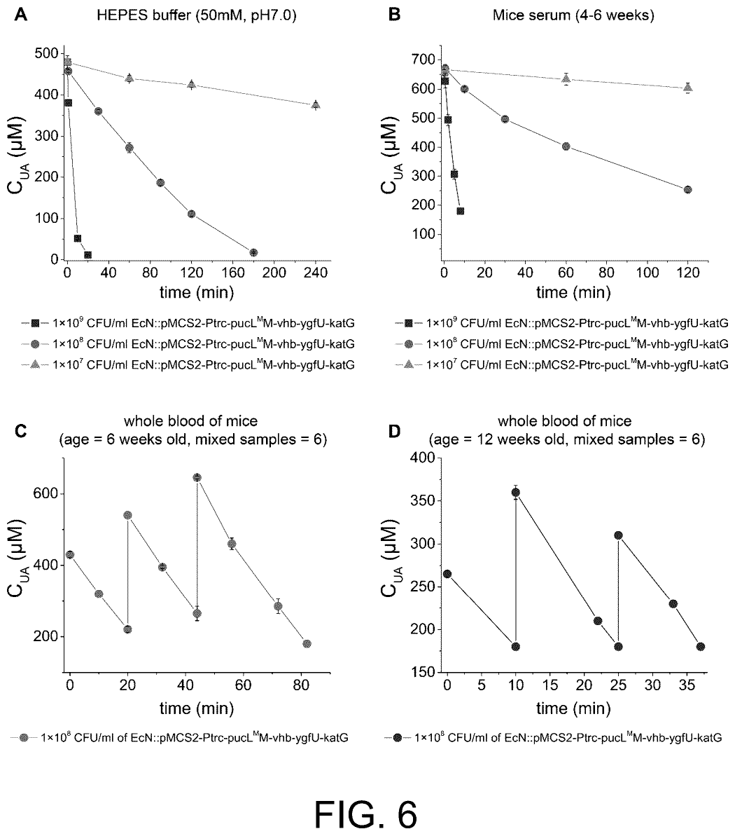

EcN cells have been intravenously injected to treat tumors. Whether the injection of EcN::pucL^{M}M-vhb-ygfU-katG cells into the blood could treat hyperuricemia was tested. First, the induced cells rapidly degraded with added UA in HEPES buffer (pH=7.0) (Control) and commercial mice serum (A&B of FIG. 6). Second, the whole blood samples of mice were divided into two groups by age: young group (6 weeks old) and old group (12 weeks old), and a defined concentration of UA was added. When the EcN::pucL^{M}M-vhb-ygfU-katG cells (1×10⁸ CFU/mL) were added, they rapidly degraded UA in both groups (C&D of FIG. 6). 320 μM UA was repeatedly added in the whole blood samples, and the UA degradation rates were not altered (C&D of FIG. 6).

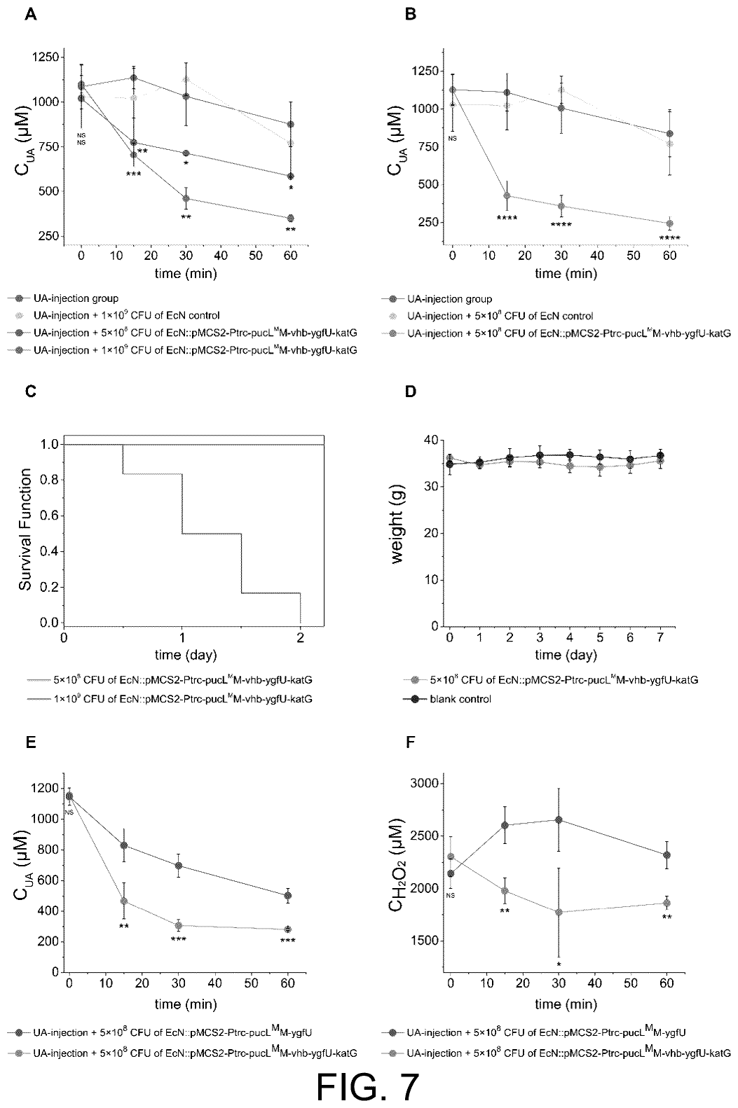

Third, 100 μL of the induced cells at 5×10⁸ CFU and 1×10⁹ CFU were released into the UA-injection hyperuricemia mice at the same time. The serum UA level in the treated group decreased faster than the untreated group (A of FIG. 7). More engineered EcN offered faster UA degradation (A of FIG. 7). However, 1×10⁹ CFU cells were not suitable for hyperuricemia treatment, since injection of high doses of the EcN cells increased the mortality of mice in 24 hours (C of FIG. 7). Mice with 100-μL injection of 5×10⁸ CFU cells were in good condition, no mouse was dead, and the weight was not lost even after 7 days of feeding (C&D of FIG. 7). Fourth, after 5×10⁸ CFU engineered EcN strains were injected into the vein of the mice 10 hours, UA solution was then injected into the vessel at a dose of 70 mg/kg to induce hyperuricemia. The UA level decreased much faster in the injection group than the control group without the EcN strains (B of FIG. 7). Fifth, the injection of the EcN strain with vhb and katG gene also helped the strain to degrade UA and remove H₂O₂ faster than the injection of the EcN strain without vhb and katG gene (E&F of FIG. 7).

It should be noted that the above description is only preferred embodiments of the present invention and should not be used to limit the invention. Although the above embodiments have been described in detail concerning the preceding embodiments, those skilled in the art can still modify the technical solutions described in the preceding embodiments or substitute some of them with equivalent alternatives. Any modifications, equivalent substitutions, improvements, etc. made within the spirit and principles of the present invention should be included within the scope of the invention. Although specific embodiments of the present invention have been described, it should be understood that they do not limit the scope of protection of the present invention. Those skilled in the art should understand that various modifications or variations that can be made without inventive effort based on the technical solutions of the present invention are still within the scope of protection of the present invention.

1. An engineered probiotic for degrading uric acid, wherein the probiotic is a derivative of Escherichia coli, and wherein one or more exogenous genes selected from a urate oxidase gene, a urate transporter gene, a hemoglobin gene, and a catalase gene are integrated into its genome.

2. The engineered probiotic for degrading uric acid according to claim 1, wherein the derivative of Escherichia coli is a derivative of Escherichia coli Nissle 1917.

3. The engineered probiotic for degrading uric acid according to claim 1, wherein the urate oxidase gene is specifically pucL^{M} and pucM, whose nucleotide sequences are shown in SEQ ID NO: 1 and SEQ ID NO: 2;
the urate transporter gene is specifically ygfU, whose nucleotide sequence is shown in SEQ ID NO: 3;
the hemoglobin gene is specifically vhb, whose nucleotide sequence is shown in SEQ ID NO: 4;
the catalase gene is specifically katG, whose nucleotide sequence is shown in SEQ ID NO: 5.

4. A method for the engineered probiotic for degrading uric acid according to claim 1, wherein the method comprises: introducing any one or more of the exogenous genes selected from the urate oxidase gene, the urate transporter gene, the hemoglobin gene, and the catalase gene into Escherichia coli.

5. The method according to claim 4, wherein the method comprises: constructing a recombinant expression vector, and transferring the recombinant expression vector into Escherichia coli Nissle 1917 for expression.

6. The method according to claim 5, wherein the recombinant expression vector is obtained by effectively connecting any one or more of the genes to an expression vector, wherein the expression vector is any one or more of a viral vector, a plasmid, a phage, a phagemid, a cosmid, a fosmid, or an artificial chromosome;
the urate oxidase gene is specifically pucL^{M} and pucM, whose nucleotide sequences are shown in SEQ ID NO: 1 and SEQ ID NO: 2;
the urate transporter gene is specifically ygfU, whose nucleotide sequence is shown in SEQ ID NO: 3;
the hemoglobin gene is specifically vhb, whose nucleotide sequence is shown in SEQ ID NO: 4;
the catalase gene is specifically katG, whose nucleotide sequence is shown in SEQ ID NO: 5;
wherein, a Ptrc promoter is used to express the above genes.

7. The method according to claim 5, wherein the transferring the recombinant expression vector into Escherichia coli Nissle 1917 is carried out by a transformation method comprises biologically acceptable direct transformation methods comprising gene gun method, electroporation method, ultrasound method, microinjection method, PEG method, or indirect transformation methods comprising DNA viral vector-mediated method and agrobacterium-mediated method.

8. A method for degrading uric acid, comprising the engineered probiotic according to claim 1.

9. A pharmaceutical composition, comprising the engineered probiotic according to claim 1 as an active ingredient.

10. A method for treating diseases related to elevated uric acid levels, wherein the method comprises administering to a subject a therapeutically effective dose of the engineered probiotic according to claim 1 and/or a pharmaceutical composition containing the engineered probiotic.

11. The method for constructing according to claim 5, wherein the recombinant expression vector is a plasmid of bacteria.

12. The method for constructing according to claim 11, wherein the plasmid of bacteria is pBBR1MCS-2.

13. The method for constructing according to claim 7, wherein the transformation method is carried out by the electroporation method.

14. The pharmaceutical composition according to claim 9, wherein the pharmaceutical composition further comprises one or more pharmaceutically acceptable carriers, excipients, and/or diluents.


SEQUENCE LISTING (6 sequences)

SEQ ID NO: 1 - DNA, 1485 bp, synthetic construct
  source 1..1485: mol_type = other DNA; note = nucleotide sequence of pucLM
atgtttaccatggatgatctgaaccagatggatacccagaccctgaccgataccctgggcagcatttttgaacatagcagctggattgcggaacgcagcgcggcgttacgtccttttagcagcctgagcgatctgcatcgcaaaatgaccggcattgtgaaagcggcggatcgcgaaacccagctggatctgattaaaaagcatccgcgcctgggcaccaagaaaaccatgagcgatgatagcgtgcgcgaacagcagaacgcgggcttaggcaaactggaacagcaggaatatgaagaatttctgatgctgaacgaacattactatgaccgctttggctttccgtttattctggcggtgaaaggcaaaaccaaacaggatattcatcaggcgctgctggcccgcctggaaagcgaacgtgaaaccgaatttcagcaggcgctgattgaaatttatcgcattgcgcgttttcgcctggcggatattattaccgaaaaaggcgaaacccagatgaaacgcaccatgagctatggcaaaggcaacgtgtttgcgtatcgcacctatctgaaaccgctgaccggcgtgaaacagattccggaaagcagctttgcgggccgcgataacaccgtggtgggcgttgttgtgacctgcgaaattggcggcgaagcgtttctgccgagctttaccgatggcgataacaccctggtggtggcgaccgatagcatgaaaaactttattcagcgccatctggcgagctatgaaggcaccaccaccgaaggctttctgcattatgtggcgcatcgctttctggatacctatagccatatggataccattaccctgaccggcgaagatattccgtttgaagcgatgccggcgtatgaagaaaaagaactgagcaccagccgcctggtgtttcgccgctcacgtaatgaacgtagccgtagcgttctgaaagcggaacgcagcggtaacaccattaccattaccgaacagtatagcgaaattatggatctgcagctggtgaaagtgagcggcaacagctttgtgggctttattcgcgatgaatataccaccctgccggaagatggcaaccgcccgctgtttgtgtatctgaacattagctggcagtatgaaaacaccaacgatagctatgcgagcgatccggcccgctatgtggcggcggaacaagttcgtgatctggcgagcaccgtgtttcatgaactggaaaccccgagcattcagaacctgatttatcatattggctgccgcattctggcccgctttccgcagttaaccgatgtgagctttcgcagccagaaccatacctgggataccgtggtggaagaaattccgggcagcaaaggcaaagtgtataccgaaccgcgcccgccgtatggctttcagcattttaccgtgacccgcgaagatgcggaaaaagaaaaacagaaagcggcggaaaaatgccgcagcctgaaagcgtaa

SEQ ID NO: 2 - DNA, 345 bp, synthetic construct
  source 1..345: mol_type = other DNA; note = nucleotide sequence of PucM
atgggcaaactgaccacccatattctggatctgacctgcggcaaaccggcggcgaatgtgaaaattggcctgaaacgcctgggcgaaagcattatgaaagaagtgtataccaacaacgacggccgcgtggatgtgccgttactggcgggtgaagaattaatgagcggcgaatatgtgatggaatttcatgcgggcgattattttgcgagcaaaaacatgaacgcggcggatcagccgtttctgaccattgtgaccgtgcgctttcagctggcggatccggatgcgcattatcatattccgctgctgctgagcccgtttggctatcaggtgtatcgcggcagctaa

SEQ ID NO: 3 - DNA, 1449 bp, Escherichia coli
  source 1..1449: mol_type = genomic DNA; note = Gene ygfU
atgagcgccatagattcccaacttccctcatcttctgggcaagaccgcccaactgatgaggttgaccgcatattatcaccaggaaagctgatcatactcggtctgcaacacgtccttgtcatgtacgcaggtgcagtcgctgttcctcttatgattggtgaccgactgggcctctcaaaagaagctattgcgatgctcattagctcggatctcttttgctgcgggatcgtcacattattgcaatgtatcggtatcggccgctttatggggatccgcctgccggtgattatgtcggtgacctttgctgctgtaacaccaatgatagccattgggatgaacccggatatcggcctgctggggatatttggtgccactatcgccgcgggttttatcaccacattattagcgccacttatcggtcgcttgatgcctttattcccgccactggttaccggtgtggttattacttctatcgggcttagcatcattcaggtgggtattgactgggccgccggaggtaaagggaatccgcaatatggtaatcccgtttatttaggtatctcctttgccgtcttaatttttatcttgctcattactcgctatgcgaaaggatttatgtccaacgtcgccgtattactggggattgtatttggctttttactttcgtggatgatgaatgaagtcaatttatccgggctacatgatgcttcatggtttgcgattgttacgccgatgtcgtttggtatgccgattttcgatcccgtttccattctgaccatgactgccgtgttaatcatcgtgtttatcgagtcaatggggatgttcctggcactgggtgaaatagtcggtcgtaaactctcttcgcacgatattattcgcgggctgcgtgtcgatggcgtagggacaatgataggcggcacgtttaacagcttcccccacacgtcattttctcaaaacgttggcctggttagcgtgacgcgcgttcatagccgctgggtgtgtatttcttcgggaattatattaatcctgtttggcatggtgccaaaaatggcggtgctggtagcctccattccgcaatttgtgctgggcggcgctggtctagtgatgttcggcatggtactggcgacagggattcgaattctgtcgcgctgtaactacaccaccaaccgttacaacctctatattgtggcgatcagtctcggcgttggcatgactccgacgctctctcacgatttcttttctaagttaccggccgtactgcaaccgctgctacatagcggcattatgctcgcaacccttagcgccgttgtgctgaacgtcttctttaatggctatcagcatcatgctgacctggtgaaggaatccgtctctgataaagatttaaaagtcaggacagtacgtatgtggcttctgatgcgcaagctgaagaaaaatgagcatggagaataa

SEQ ID NO: 4 - DNA, 441 bp, synthetic construct
  source 1..441: mol_type = other DNA; note = nucleotide sequence of Vhb
atgctggatcagcagaccattaacattattaaagccaccgttccggttctgaaagaacacggcgttaccattaccaccaccttttataaaaatctgttcgccaaacacccggaagttcgcccgctgtttgatatgggtcgccaggagagcctggaacagcctaaagccctggcaatgaccgttctggccgccgcacagaatattgaaaacctgccggcaattctgcctgcggtgaaaaagattgccgttaaacattgtcaggcaggagttgcagcagcacattatcctattgtgggccaggaactgctgggtgcaatcaaagaagtgctgggtgatgcagcaaccgatgatatcctggatgcatggggcaaagcctatggtgttattgcagatgtttttattcaggttgaagcagatctgtatgcacaggcagtggagtaa

SEQ ID NO: 5 - DNA, 2181 bp, Escherichia coli
  source 1..2181: mol_type = genomic DNA; note = Gene katG
atgagcacgtcagacgatatccataacaccacagccactggcaaatgcccgttccatcagggcggtcacgaccagagtgcgggggcgggcacaaccactcgcgactggtggccaaatcaacttcgtgttgacctgttaaaccaacattctaatcgttctaacccactgggtgaggactttgactaccgcaaagaattcagcaaattagattactacggcctgaaaaaagatctgaaagccctgttgacagaatctcaaccgtggtggccagccgactggggcagttacgccggtctgtttattcgtatggcctggcacggcgcggggacttaccgttcaatcgatggacgcggtggcgcgggtcgtggtcagcaacgttttgcaccgctgaactcctggccggataacgtaagcctcgataaagcgcgtcgcctgttgtggccaatcaaacagaaatatggtcagaaaatctcctgggccgacctgtttatcctcgcgggtaacgtggcgctagaaaactccggcttccgtaccttcggttttggtgccggtcgtgaagacgtctgggaaccggatctggatgttaactggggtgatgaaaaagcctggctgactcaccgtcatccggaagcgctggcgaaagcaccgctgggtgcaaccgagatgggtctgatttacgttaacccggaaggcccggatcacagcggcgaaccgctttctgcggcagcagctatccgcgcgaccttcggcaacatgggcatgaacgacgaagaaaccgtggcgctgattgcgggtggtcatacgctgggtaaaacccacggtgccggtccgacatcaaatgtaggtcctgatccagaagctgcaccgattgaagaacaaggtttaggttgggcgagcacttacggcagcggcgttggcgcagatgccattacctctggtctggaagtagtctggacccagacgccgacccagtggagcaactatttcttcgagaacctgttcaagtatgagtgggtacagacccgcagcccggctggcgcaatccagttcgaagcggtagacgcaccggaaattatcccggatccgtttgatccgtcgaagaaacgtaaaccgacaatgctggtgaccgacctgacgctgcgttttgatcctgagttcgagaagatctctcgtcgtttcctcaacgatccgcaggcgttcaacgaagcctttgcccgtgcctggttcaaactgacgcacagggatatggggccgaaatctcgctacatcgggccggaagtgccgaaagaagatctgatctggcaagatccgctgccgcagccgatctacaacccgaccgagcaggacattatcgatctgaaattcgcgattgcggattctggtctgtctgttagtgagctggtatcggtggcctgggcatctgcttctaccttccgtggtggcgacaaacgcggtggtgccaacggtgcgcgtctggcattaatgccgcagcgcgactgggatgtgaacgccgcagccgttcgtgctctgcctgttctggagaaaatccagaaagagtctggtaaagcctcgctggcggatatcatagtgctggctggtgtggttggtgttgagaaagccgcaagcgccgcaggtttgagcattcatgtaccgtttgcgccgggtcgcgttgatgcgcgtcaggatcagactgacattgagatgtttgagctgctggagccaattgctgacggtttccgtaactatcgcgctcgtctggacgtttccaccaccgagtcactgctgatcgacaaagcacagcaactgacgctgaccgcgccggaaatgactgcgctggtgggcggcatgcgtgtactgggtggcaacttcgatggcagcaaaaacggcgtcttcactgaccgcgttggcgtattgagcaatgacttcttcgtgaacttgctggatatgcgttacgagtggaaagcgaccgacgaatcgaaagagctgttcgaaggccgtgaccgtgaaaccggcgaagtgaaatttacggccagccgtgcggatctggtgtttggttctaactccgtcctgcgtgcggtggcggaagtttacgccagtagcgatgcccacgagaagtttgttaaagacttcgtggcggcatgggtgaaagtgatgaacctcgaccgtttcgacctgctgtaa

SEQ ID NO: 6 - DNA, 12029 bp, synthetic construct
  source 1..12029: mol_type = other DNA; plasmid = pBBR1MCS-2-Ptrc-pucLM-pucM-ygfU-vhb-katG
tctagagattaaagaggagaatactagatgctggatcagcagaccattaacattattaaagccaccgttccggttctgaaagaacacggcgttaccattaccaccaccttttataaaaatctgttcgccaaacacccggaagttcgcccgctgtttgatatgggtcgccaggagagcctggaacagcctaaagccctggcaatgaccgttctggccgccgcacagaatattgaaaacctgccggcaattctgcctgcggtgaaaaagattgccgttaaacattgtcaggcaggagttgcagcagcacattatcctattgtgggccaggaactgctgggtgcaatcaaagaagtgctgggtgatgcagcaaccgatgatatcctggatgcatggggcaaagcctatggtgttattgcagatgtttttattcaggttgaagcagatctgtatgcacaggcagtggagtaaaaagaggggaaatactagatgagcgccatagattcccaacttccctcatcttctgggcaagaccgcccaactgatgaggttgaccgcatattatcaccaggaaagctgatcatactcggtctgcaacacgtccttgtcatgtacgcaggtgcagtcgctgttcctcttatgattggtgaccgactgggcctctcaaaagaagctattgcgatgctcattagctcggatctcttttgctgcgggatcgtcacattattgcaatgtatcggtatcggccgctttatggggatccgcctgccggtgattatgtcggtgacctttgctgctgtaacaccaatgatagccattgggatgaacccggatatcggcctgctggggatatttggtgccactatcgccgcgggttttatcaccacattattagcgccacttatcggtcgcttgatgcctttattcccgccactggttaccggtgtggttattacttctatcgggcttagcatcattcaggtgggtattgactgggccgccggaggtaaagggaatccgcaatatggtaatcccgtttatttaggtatctcctttgccgtcttaatttttatcttgctcattactcgctatgcgaaaggatttatgtccaacgtcgccgtattactggggattgtatttggctttttactttcgtggatgatgaatgaagtcaatttatccgggctacatgatgcttcatggtttgcgattgttacgccgatgtcgtttggtatgccgattttcgatcccgtttccattctgaccatgactgccgtgttaatcatcgtgtttatcgagtcaatggggatgttcctggcactgggtgaaatagtcggtcgtaaactctcttcgcacgatattattcgcgggctgcgtgtcgatggcgtagggacaatgataggcggcacgtttaacagcttcccccacacgtcattttctcaaaacgttggcctggttagcgtgacgcgcgttcatagccgctgggtgtgtatttcttcgggaattatattaatcctgtttggcatggtgccaaaaatggcggtgctggtagcctccattccgcaatttgtgctgggcggcgctggtctagtgatgttcggcatggtactggcgacagggattcgaattctgtcgcgctgtaactacaccaccaaccgttacaacctctatattgtggcgatcagtctcggcgttggcatgactccgacgctctctcacgatttcttttctaagttaccggccgtactgcaaccgctgctacatagcggcattatgctcgcaacccttagcgccgttgtgctgaacgtcttctttaatggctatcagcatcatgctgacctggtgaaggaatccgtctctgataaagatttaaaagtcaggacagtacgtatgtggcttctgatgcgcaagctgaagaaaaatgagcatggagaataactagagaaagaggagaaatactagatgagcacgtcagacgatatccataacaccacagccactggcaaatgcccgttccatcagggcggtcacgaccagagtgcgggggcgggcacaaccactcgcgactggtggccaaatcaacttcgtgttgacctgttaaaccaacattctaatcgttctaacccactgggtgaggactttgactaccgcaaagaattcagcaaattagattactacggcctgaaaaaagatctgaaagccctgttgacagaatctcaaccgtggtggccagccgactggggcagttacgccggtctgtttattcgtatggcctggcacggcgcggggacttaccgttcaatcgatggacgcggtggcgcgggtcgtggtcagcaacgttttgcaccgctgaactcctggccggataacgtaagcctcgataaagcgcgtcgcctgttgtggccaatcaaacagaaatatggtcagaaaatctcctgggccgacctgtttatcctcgcgggtaacgtggcgctagaaaactccggcttccgtaccttcggttttggtgccggtcgtgaagacgtctgggaaccggatctggatgttaactggggtgatgaaaaagcctggctgactcaccgtcatccggaagcgctggcgaaagcaccgctgggtgcaaccgagatgggtctgatttacgttaacccggaaggcccggatcacagcggcgaaccgctttctgcggcagcagctatccgcgcgaccttcggcaacatgggcatgaacgacgaagaaaccgtggcgctgattgcgggtggtcatacgctgggtaaaacccacggtgccggtccgacatcaaatgtaggtcctgatccagaagctgcaccgattgaagaacaaggtttaggttgggcgagcacttacggcagcggcgttggcgcagatgccattacctctggtctggaagtagtctggacccagacgccgacccagtggagcaactatttcttcgagaacctgttcaagtatgagtgggtacagacccgcagcccggctggcgcaatccagttcgaagcggtagacgcaccggaaattatcccggatccgtttgatccgtcgaagaaacgtaaaccgacaatgctggtgaccgacctgacgctgcgttttgatcctgagttcgagaagatctctcgtcgtttcctcaacgatccgcaggcgttcaacgaagcctttgcccgtgcctggttcaaactgacgcacagggatatggggccgaaatctcgctacatcgggccggaagtgccgaaagaagatctgatctggcaagatccgctgccgcagccgatctacaacccgaccgagcaggacattatcgatctgaaattcgcgattgcggattctggtctgtctgttagtgagctggtatcggtggcctgggcatctgcttctaccttccgtggtggcgacaaacgcggtggtgccaacggtgcgcgtctggcattaatgccgcagcgcgactgggatgtgaacgccgcagccgttcgtgctctgcctgttctggagaaaatccagaaagagtctggtaaagcctcgctggcggatatcatagtgctggctggtgtggttggtgttgagaaagccgcaagcgccgcaggtttgagcattcatgtaccgtttgcgccgggtcgcgttgatgcgcgtcaggatcagactgacattgagatgtttgagctgctggagccaattgctgacggtttccgtaactatcgcgctcgtctggacgtttccaccaccgagtcactgctgatcgacaaagcacagcaactgacgctgaccgcgccggaaatgactgcgctggtgggcggcatgcgtgtactgggtggcaacttcgatggcagcaaaaacggcgtcttcactgaccgcgttggcgtattgagcaatgacttcttcgtgaacttgctggatatgcgttacgagtggaaagcgaccgacgaatcgaaagagctgttcgaaggccgtgaccgtgaaaccggcgaagtgaaatttacggccagccgtgcggatctggtgtttggttctaactccgtcctgcgtgcggtggcggaagtttacgccagtagcgatgcccacgagaagtttgttaaagacttcgtggcggcatgggtgaaagtgatgaacctcgaccgtttcgacctgctgtaagttaaaattcgcgttaaatttttgttaaatcagctcattttttaaccaataggccgactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacccccatgggcaaatattatacgcaaggcgacaaggtgctgatgccgctggcgattcaggttcatcatgccgtttgtgatggcttccatgtcggcagaatgcttaatgaattacaacagtttttatgcatgcgccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacactttaactttaaggaggagatataccatgtttaccatggatgatctgaaccagatggatacccagaccctgaccgataccctgggcagcatttttgaacatagcagctggattgcggaacgcagcgcggcgttacgtccttttagcagcctgagcgatctgcatcgcaaaatgaccggcattgtgaaagcggcggatcgcgaaacccagctggatctgattaaaaagcatccgcgcctgggcaccaagaaaaccatgagcgatgatagcgtgcgcgaacagcagaacgcgggcttaggcaaactggaacagcaggaatatgaagaatttctgatgctgaacgaacattactatgaccgctttggctttccgtttattctggcggtgaaaggcaaaaccaaacaggatattcatcaggcgctgctggcccgcctggaaagcgaacgtgaaaccgaatttcagcaggcgctgattgaaatttatcgcattgcgcgttttcgcctggcggatattattaccgaaaaaggcgaaacccagatgaaacgcaccatgagctatggcaaaggcaacgtgtttgcgtatcgcacctatctgaaaccgctgaccggcgtgaaacagattccggaaagcagctttgcgggccgcgataacaccgtggtgggcgttgttgtgacctgcgaaattggcggcgaagcgtttctgccgagctttaccgatggcgataacaccctggtggtggcgaccgatagcatgaaaaactttattcagcgccatctggcgagctatgaaggcaccaccaccgaaggctttctgcattatgtggcgcatcgctttctggatacctatagccatatggataccattaccctgaccggcgaagatattccgtttgaagcgatgccggcgtatgaagaaaaagaactgagcaccagccgcctggtgtttcgccgctcacgtaatgaacgtagccgtagcgttctgaaagcggaacgcagcggtaacaccattaccattaccgaacagtatagcgaaattatggatctgcagctggtgaaagtgagcggcaacagctttgtgggctttattcgcgatgaatataccaccctgccggaagatggcaaccgcccgctgtttgtgtatctgaacattagctggcagtatgaaaacaccaacgatagctatgcgagcgatccggcccgctatgtggcggcggaacaagttcgtgatctggcgagcaccgtgtttcatgaactggaaaccccgagcattcagaacctgatttatcatattggctgccgcattctggcccgctttccgcagttaaccgatgtgagctttcgcagccagaaccatacctgggataccgtggtggaagaaattccgggcagcaaaggcaaagtgtataccgaaccgcgcccgccgtatggctttcagcattttaccgtgacccgcgaagatgcggaaaaagaaaaacagaaagcggcggaaaaatgccgcagcctgaaagcgtaatttaactttaaggaggagatataccatgggcaaactgaccacccatattctggatctgacctgcggcaaaccggcggcgaatgtgaaaattggcctgaaacgcctgggcgaaagcattatgaaagaagtgtataccaacaacgacggccgcgtggatgtgccgttactggcgggtgaagaattaatgagcggcgaatatgtgatggaatttcatgcgggcgattattttgcgagcaaaaacatgaacgcggcggatcagccgtttctgaccattgtgaccgtgcgctttcagctggcggatccggatgcgcattatcatattccgctgctgctgagcccgtttggctatcaggtgtatcgcggcagctaa